(19) United States
(12) Patent Application Publication
(10) Pub. No.: US 2004/0197824 A1
(43) Pub. Date: Oct. 7, 2004

(54) Method for selective radioactive marking of peptides

(76) Inventors: Norman Koglin, Leipzig (DE); Manja Lang, Leipzig (DE); Annette G. Beck-Sickinger, Leipzig (DE)

Correspondence Address: Horst M. Kasper, 13 Forest Drive, Warren, NJ, 07059, US

(21) Appl. No.: 10/653,779

(22) Filed: Sep. 2, 2003

Related U.S. Application Data
(60) Provisional application No. 60/407,179, filed on Aug. 30, 2002.

(51) Int. Cl. G01N 33/53

(52) U.S. Cl. 435/7.1; 530/403

(57) ABSTRACT

The invention relates to a method for selective radio marking of peptides. It is the object to present a generally applicable method for the production of radio ligands. The object is accomplished by the use of photo labile protective groups in peptide synthesis, wherein the photo labile groups block pre-given amino groups for such time until the desired derivative formation of a specific amino group is performed with a ³H-containing group and the protective groups are split off by UV radiation.

The invention relates to a method for selective radioactive marking of peptides, which method is generally applicable and can be performed advantageously also at a microscale.

Always new and selectively marked bio molecules are required in research, medicine, and pharmaceutical industry for performing of bio activity studies especially in the post genome age. Thus one requires marked and thereby easily detectable peptides for the investigation of the interaction of peptides for example with peptide receptors in binding studies and for structural infection relationships. This marking however is not permitted to limit the biological activity. Here radioactively marked peptides are associated with the advantage relative to peptides marked by fluorescence that they are detectable with high sensitivity and change the biological properties of the bio molecules to a lesser extent.⁽¹⁾

The isotopes employed most frequently in protein biochemistry and peptide chemistry are ¹²⁵iodine (¹²⁵I), ³⁵sulphur (³⁵S) and tritium (³H). The half value times of ¹²⁵I and ³⁵S amount to 60 or, respectively, 87 days, the half value time of ³H in contrast is 12.4 years. Based on the clearly longer half value time of the tritium it is possible to perform stable measurements with a radio ligand over a longer time period without that corrective values would have to be introduced. In addition, the radiation energy of the individual isotopes is in part substantially different. Thus tritium is a β-radiator with a small range (0.6 cm in air) and a small radiation energy. ¹²⁵I is a γ-radiator with a high penetration capability and a very energy rich electromagnetic radiation. The required protective steps with the employment of tritium are therefore substantially less as compared with ¹²⁵I. Therefore chemical bonds can be easier irreversibly destroyed based on the stronger radiation in ¹²⁵I marked peptides. The required protective steps are therefore substantially less in the employment tritium as compared to ¹²⁵I. Based on the stronger radiation in ¹²⁵I-marked peptides, chemical bonds can therefore be easier irreversibly destroyed. This leads to undefined radioactive or non-radioactive fragments, wherein these fragments negatively influence successive investigations. The radioactive concentration decreases by radiolysis. The rate of radiolysis can be reduced however by storage at low temperatures⁽²⁾.

In general peptides can be radioactively marked in two different ways. The first way comprises the direct marking. Here the peptides are synthesized during the peptide synthesis with the radioactive marked amino acids (for example with ³⁵S-methionine) or the peptides are marked following to the synthesis with a direct reaction with the isotope. The radioactivity is incorporated into the peptide by indirect marking according to the second way. Here preferably primary amino groups (n-terminal or ε-amino groups of lysine residues) are formed into derivatives.

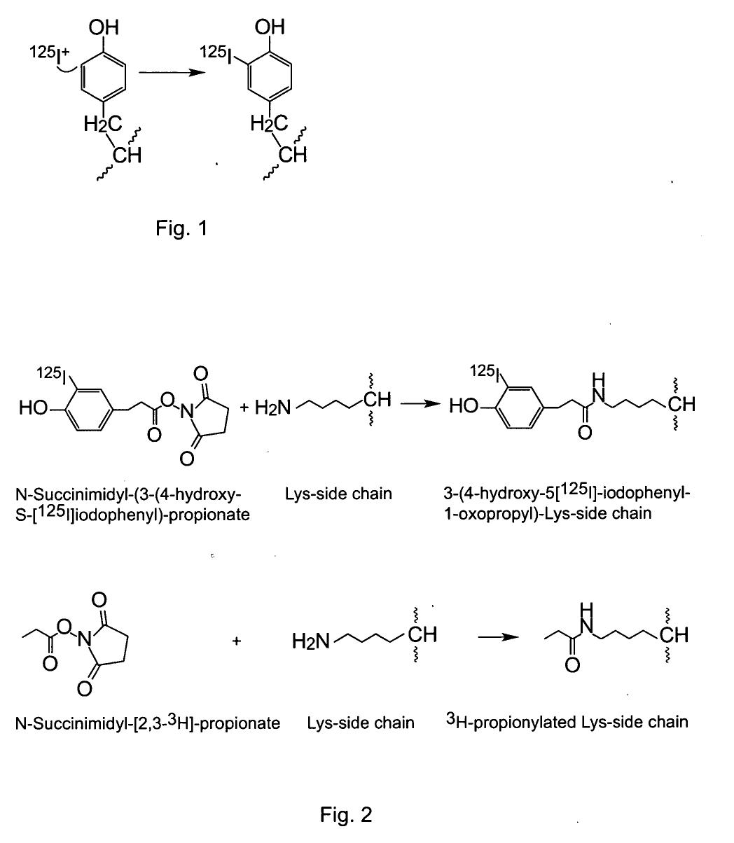

One or several atoms ¹²⁵I are introduced into the peptide directly at the His- or Tyr-side chains during the direct marking with ¹²⁵I. Here the Na¹²⁵I is oxidized to ¹²⁵I₂(I⁺I⁻) by chloramine-T or by the H₂O₂/lacto-peroxidase system. One or two ¹²⁵I⁺-atoms for each Tyr- or His-side chain are incorporated into the peptide (FIG. 1) in the following by electrophilic substitution. Not only one side chain is marked in case of a presence of more than one of these amino acids in the peptide but instead here different residues in the peptide are turned into derivatives. An inhomogeneous product is generated thereby, wherein the inhomogeneous product requires further purification steps. This is in particular required where such side chains were modified here, which side chains are of importance for the biological recognition and which side chains cause an affinity loss based on the introduction of the relatively large iodine atom.

Tritium gas, ³H-sodium boro hydride or ³H-methyl iodide can be employed for the direct marking of peptides with tritium. The marking is performed by isotope exchange (³H-gas, ³H-water, ³H-acetic acid, metal catalysis), chemical synthesis, biochemical methods or conjugation. The exchange reactions are performed in general at temperatures of more than 100° C. in the presence of a catalyst. Unsaturated compounds (C—C double bonds, ketones) are reduced upon employment of tritium gas. The handling with these reagents can only be performed in especially equipped laboratories and under heavy safety requirements. This method for the tritium marking of peptides is less suitable because of the particular reaction conditions and the required functional groups.

Mostly primary amino groups such as for example the N-terminal or the ε-amino groups of the Lys-side chains are turned into derivatives by Bolton-Hunter reaction in the case of the indirect marking. The Bolton-Hunter reagents belong to the class of the n-hydroxy-succinimide-(NHS)-ester, which transfer acyl residues to amino groups⁽³⁾. Frequently employed and commercially available radioactive acyl residues are provided in the way of their NHS-ester, for example for ¹²⁵I the 3-(4-hydroxy-5-[¹²⁵I] iodo-phenyl)-proprionyl-group or for ³H the [2,3-³H]-proprionyl-group. The marking of a Lys-side chain is illustrated in FIG. 24 for these two NHS-ester.

The indirect radioactive marking of peptides can be performed

a.) with a side chain protected peptide still bound to the resin after the synthesis or

b.) also with a free peptide in solution.

The advantage of a marking at the resin comprises the possibility of selectively deprotecting amino acid side chains and to turn the amino acids side chains into derivatives. This advantage however is lost in the following to the synthesis by in part expensive work steps under radioactive conditions. Furthermore, only charges starting with the mg region can be worked with in connection with peptides at a resin. This requires however substantial amounts of radioactivity and causes very high costs. Since a purification step of the peptides is not possible prior to the radioactive marking at the resin, in part high losses of radioactivity can occur during the performing of these work and apparatus intensive reprocessing steps (for example in case of a preparative high-pressure liquid chromatography HPLC). A large part of the employed radioactivity gets lost again by also marked erroneous sequences in particular in connection with difficult to synthesize peptide sequences and thus more heavily contaminated raw products during the reprocessing. The employment of large quantities of radioactivity is in addition a high expenditure point, requires special conditions, represents in addition a higher risk of health and can endanger the environment based on a larger volume of waste. The employment of larger radioactivity volumes requires in addition a high expenditure pertaining to apparatus especially for the reprocessing and the analysis of the radioactively marked peptides.

In most cases however already a few micrograms of a high affinity radio ligand are sufficient for numerous investigations for biological studies. Furthermore, frequently different and at different positions marked peptides are required in particular in the case of the new receptor-ligand-interaction studies. Therefore it is reasonable in the frame of research projects to prepare and to employ initially only smaller quantities of a radioactive bio molecule for testing.

The indirect marking of peptides in solution is also associated with advantages and disadvantages. It is an advantage that already very small amounts (μg region) can be handled. Reactions, which are performed with free peptide in solution, however can be associated with the disadvantage that in particular in case of longer peptides under certain circumstances several reactive groups are freely present. This is a particular problem when several free amino groups are present in the peptide sequence. This is for example the case where the peptides in addition to their mostly free n-terminal group have available one or several Lys-residues and these residues in each case cannot be present in modified form for a full biological activity. Based on the different pK-values of the ε-amino group of Lys and of the alpha-amino group of the free N-terminal group in fact preferably at one position the the marking can be performed by selection of suitable reaction conditions (pH-Value), however this succeeds in most cases not completely and their result inhomogeneous radio ligands⁽⁴⁾. If in addition several Lys-side chains are present, then the peptide is non-selectively marked in addition at several positions and thus can be turned into derivatives also at radicals important for the biological activity. The non-selectively marked radio ligands prepared in this manner have to be purified in the following. This is associated with losses relative to the desired radio ligand and thereby with higher costs and expenditures. In addition, under certain circumstances it can be very difficult in particular in connection with larger peptides to efficiently separate isomers and double or, respectively, multiply marked peptides from each other. If the non-selectively marked peptides cannot be separated completely from each other, then the biological activity can be influenced in biological studies and the quantitative force of conclusions of biological assays can be substantially reduced.

It is an object of the present invention to selectively mark peptides independent of the sequence by an indirect radioactive marking in solution. The amount of peptide to be employed here can already be handled on a μg-scale and is not bound to minimum amounts caused by the method. Homogeneous radio ligands are obtained in a quality which corresponds to the high requirements of quantitative biological investigations. The radioactivity amount to be charged can be controlled corresponding to the desired amount of radio ligand based on this method compatible already in a micro-scale. A further advantage of the method comprises the reduced number of work intensive, radioactive processing steps and in the performability of the elective marking associated with small apparatus and analytical expenditures. This method therefore can be more cost-effective and more simply performed as compared to conventional methods.

It is an object of the invention to furnish a technically simple manipulable method which allows the marking of peptides at a certain amino group. Here the marking is to be performed at peptides which are present in solution and which can exhibit also additional amino functions in addition to the amino group to be marked. The additional amino functions however are to be temporarily protected during the marking and are to be freely present again after the marking reaction. The obtained peptides are to be employed as radio ligands and are to lead to new results and new knowledge in research.

The scientific recognition that photo labile protective groups can be employed for the protection of the amino groups not to be marked is the basis of the present invention, wherein the photo labile protective groups assure the selective marking based on their presence in the molecule and wherein the photo labile protective groups can be simply removed again after the marking reaction. For this purpose of particular process course of the synthesis of peptides has to be found wherein the process course allows to occupy all positions in the molecule not intended for marking and only to release the predetermined position for marking and finally to mark the predetermined position.

The Invention is realized as is indicated in the claims.

The first step is the introduction of photo labile protective groups at defined amino groups in the peptide.

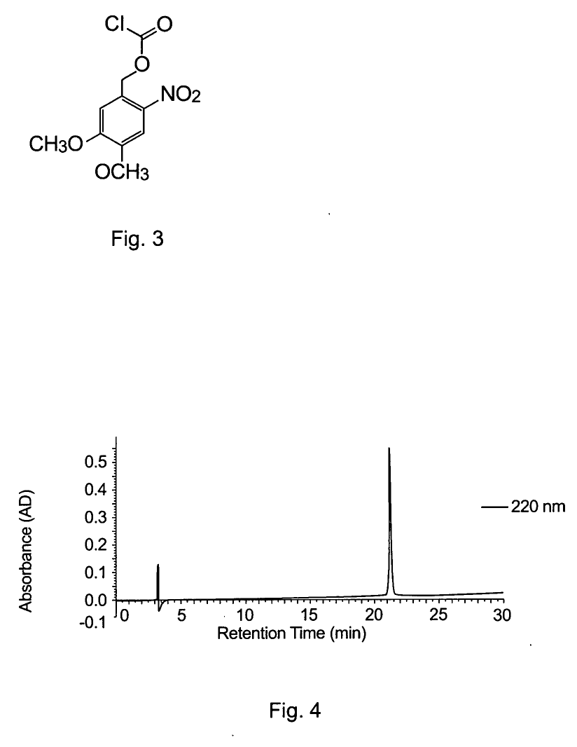

The peptide is initially synthesized by way of solid phase peptide synthesis with the orthogonal Fmoc/tert.butyl protective group strategy at a milligram scale (13.5 μmole)⁽⁵⁾. In case several free amino groups are present in the peptide, for example the N-terminal and several Lys-residues, then one would obtain an inhomogeneous product upon a following marking of the unprotected peptide in solution. In order to avoid this, those amino groups not to be marked radioactively are protected temporarily with the photo labile Nvoc-protective group (FIG. 3). This protective group can be easily removed by ultraviolet radiation after the radioactive marking.

A commercially available Lys-derivative with 1-(4,4-di-methyl-2,6-dioxo-cyclohex-1-ylidene) ethyl-(Dde)-protected amino function (Novabiochem) in the side chain is employed (schema 1) during the synthesis for ε-amino groups of Lys-side chains, wherein the Lys-side chains are still to be protected during the marking reaction and are to be present freely during later biological investigations. The Dde-protective group of the Lys-side chain(s) can be selectively removed immediately at the resin with hydrazine after the synthesis⁽⁹⁾. The amino group to be later modified, a certain Lys-side chain or the N-terminal group remain then still protected with the acid labile Boc-protective group. For this purpose an α-Fmoc-protected amino acid is to be employed in case of a marking of the N-terminal group in the last coupling step of the peptide synthesis (schema 2). If however in contrast an α-Fmoc-protected amino acid was employed in the last coupling step, then the n-terminal group is already present freely after the synthesis at the resin. All freely present amino groups are furnished simultaneously with the photo labile Nvoc-protective group after the splitting off of the Dde-protective group(s).

The introduction of the Nvoc-group at all relevant positions at the polymer carrier is new according to the knowledge and experience of the applicants. The introduction is better and simpler performed as this would have been the case upon use of the previously N^{ε}-Nvoc-protected Lys-derivatives during synthesis⁽¹⁰⁾. Such derivatives are in addition not available commercially and would have to be synthesized initially.

If the N-terminal group is also not to be influenced by the later marking, then also the n-terminal group has to be present with photo labile protection. For this purpose also in each case required N^{α}-Nvoc-protected amino acid would have to be previously synthesized and would have to be employed in the last coupling step. The shown method for the simultaneous introduction of the Nvoc-group at the resin at all relevant amino functions is new and does not require the production and the use of previously Nvoc-protected amino acids.

The introduction of the Nvoc-group is illustrated in more detail in the following. The Nvoc-incorporation is performed at the resin at those positions, which are to be again freely present after the radioactive marking. The Dde-protective group can be selectively removed at the resin with hydrazine from the employed N^{ε}-Dde-protected Lys-derivatives during the synthesis⁽⁹⁾. These amino groups and possibly the N-terminal group are newly protected with introduction of the Nvoc-group. 5 equivalents 6-nitro-veratryl-oxy-carbonyl-chloride (Nvoc-CL, Sigma-Aldrich), 5 equivalents N-hydroxy-benzo-triazole (HOBt, Novabiochem), and 10 equivalents di-iso-propyl-ethyl-amine (DI-PEA, Fluka) are employed for this purpose with reference to the number of the in each case present free amino functions. N, N′-di-methyl-form amide (DMF, Biosolve) served as a solvent. The molarity of the reaction solution is to be as high as possible, the volume is to be sufficient in order to cover the resin completely. The volume of the reaction solution in each case is here dependent on the size of the peptide and amounts to about 0.5-1 ml. The also in DMF dissolved reagents are added to the resin soaked in DMF and are allowed to react under shaking for at least three hours at room temperature or overnight. After termination of the reaction, it is tested with the KAISER-test if all free amino groups have reacted⁽¹¹⁾. If this is not the case, then the reaction is performed again.

After complete reaction the resin is washed thoroughly with DMF, methanol, dichloro-methane and di-ethyl-ether and dried in vacuum. The peptide is split off in the following from the resin with 1 ml tri-fluoro-acetic acid (TFA). At the same time the acid labile side chain protective groups are removed here. A mixture of thio-anisol (Fluka) and thio-cresol (Fluka) in the ratio of 1:1 (v/v) is employed as a scavenger for the removal of the thereby occurring reactive intermediates. A mixture of ethane-di-thiol (Fluka) and thio-cresol (3:7 (v/v)) is employed as a scavenger for Cys-, Met-, and Trp-containing peptides. The ratio of TFA to scavenger is here 9:1. The peptides dissolved in TFA are separated from the polymer carrier by filtration and are precipitated by di-ethyl-ether cooled with ice. A separation of scavenger and protective group fragment is performed by centrifugation and multiple washing with cooled di-ethyl-ether. The peptides are dried in vacuum in the following and are lyophilized. If Met-radicals are present in the peptide sequence, then a following reduction of the in part to sulfoxide oxydized thio-ether-side chains with tri-methyl bromo-silane is recomended⁽¹²⁾.

The Nvoc-groups remain during the splitting off with tri-fluoro acetic acid TFA and the following preparation processing at the peptide. The amino groups, which should be present in free-form after the marking reaction, remain in this manner furthermore protected. The peptide can now be analyzed with standard technology and further be purified. The analysis is performed by way of HPLC (RP18-column with gradient elution: solvent A=0.1 percent TFA in aqua dest. and solvent B=0.08 percent TFA in aceto-nitrile ACN) and electrospray-mass spectroscopy (ESI-MS). Despite a certain stability of the Nvoc-group against daylight and room illumination it is recommended to cover the reaction vessels and to avoid an immediate and direct sun irradiation.

The second step comprises the radioactive marking.

The radioactive marking was performed with N-succino-imidyl-[2,3-³H]-propionate (Amersham). The use of other NHS-esters and radio isotopes is also possible.

The reaction can already be performed with a few microgram peptide. Since it is difficult to weigh these small amounts, a stock solution of the peptide was produced in bi-distilled water after purification with preparative HPLC and after protection with Nvoc, the desired amount (for example 2 nmole, depending on the molecule weight of the peptide about 10 μg) was removed from the stock solution and lyophilized.

The radioactive marking can now be performed both in an organic solvent such as DMF or in an aqueous buffer at neutral or slightly basic pH values. For this purpose of the Nvoc-protected peptide is dissolved in 10 μl 0.1% Di-PEA/DMF or in a slightly basic buffer. For example 10 mM phosphate buffer (pH=7.5) or, respectively, 10 mM borate buffer (pH=8.0) can be employed, and optionally aceto nitrile (ACN) can be added in case of a small solubility of the peptide. A slightly acid buffer (for example pH 6.5) can also be employed based on the low pK-value of the α-amino group in connection with a radioactive marking at the N-terminal group. A compromise exists and has to be entered between the reactivity of the amino group (stronger at higher pH values) and the stability of the NHS-ester in an aqueous medium (lower at higher pH values) in the selection of the pH value.^{(13, 14)}

The peptide is the less costly component in the marking reaction and the nucleophilic group and should the employed in a slight excess for increasing the yield. The ³H-proprionyl-NHS-ester is commercially available (Amersham). The radioactive concentration amounts to 37 MBq/ml, the specific activity amounts to 3.48 TBq/mmole. The ester is present dissolved in toluene, wherein the toluene is removed in a N₂-stream prior to the reaction. The reaction is preferably performed in silanized Eppendorf tubes, in order to keep absorptions of ester and peptide low at the wall of the vessel. In case of a double excess of peptide (2 nmole), 100 μl of the ³H-proprionyl-NHS-ester solution (1 nmole) were employed of the above recited radioactive concentration and specific activity. The peptide dissolved in the buffer or in DMF is added to the ester residue and is well mixed. The reaction lasts about 1-2 hr. at room temperature. Here also a long reaction time can be selected since no hydrolysis of the ester occurs in DMF as a solvent.

The third step comprises the splitting off of the photo labile protective group.

The charge is stored on ice for splitting off the Nvoc-group and is irradiated with ultraviolet UV-light for 30-60 minutes opened (UV-lamp: Atlas Fluotest® Forte, 366 nm, 180 Watts). The preceding catching of any ester not reacted is not necessary in an aqueous medium. After the reaction time in the aqueous medium the assumption can be made that the ester was aminolyzed or, respectively, was completely hydrolyzed by the side reaction with water and thus the ester is not any longer capable of reacting with the newly released amino groups. A nucleophilic group like glycine should be added after marking reaction in DMF in order to avoid non-selective markings based on non-reacted ester after the Nvoc-splitting off.

In order to avoid a fragmentation in particular of Trp-containing peptides by the ultraviolet UV-radiation, the UV-in section should be limited to a maximum time of 30 minutes with these peptides. The Nvoc-group is nearly quantitatively removed from the peptide also with this procedure. The splitting off is performed in an acid medium in order to avoid side reactions, in particular the formation of covalent adducts of the split off protective group with the released amino groups. For this purpose the 10 μl of the marking charge are combined with the 10 μl of a 0.1 percent aqueous TFA/ACN-solution. The ACN-content can be set here already to the successive starting conditions of the HPLC-gradient.

Finally the reprocessing is performed in the following way.

The now completely de-protected and selectively marked peptide is purified with HPLC from unreacted peptidic starting compound employed in excess, from ester- and buffer components as well as Nvoc-protective group fragments. 0.1% TFA in water and 0.08% TFA in ACN are employed as a mobile phase for the HPLC. The separation is performed by way of gradient elution (for example 30% ACN to 45% ACN over 55 minutes at the flow of 0.6 ml/minute) at a “reversed phase” (RP) C₁₈-column. 10 μl aliquots are removed from the fractions collected every minute, combined with scintillation cocktail and measured in a β-counter. The radioactivity profile is produced from these measurement results. The fractions, which correspond to the desired product based on the retention time and the radioactivity; profile, are dried in a Speed-Vac and in the following taken up in a buffer (0.15 M tri-ethyl-ammonium-phosphate/ACN=3:1, pH=3.4) and are combined.

Peptides can be selectively radioactively marked with the here presented method.

The reaction can already be performed well on a microscale.

### EXAMPLE 1

### Preparation of ³H-proprionyl-Lys⁴-NPY(³H-NPY)

The normal peptide Y (NPY) comprises 36 amino acids and contains a free amino group in the Lys⁴-side chain in addition to the N-terminal group⁽¹⁵⁾. It could be shown that a free N-terminal group is required for the full biological activity. The amino function of the Lys⁴-side chain in contrast can be modified for the production of radio ligands without loss of affinity⁽¹⁶⁾. The radio ligands are commercially available (Amersham) in the form of ¹²⁵I-NPY and ³H-NPY.

Initially N-terminal Nvoc-protected NPY was synthesized and in the following radioactively marked at the Lys⁴-side chain by reaction with ³H-proprionyl-NHS-ester for validation of the here presented marking method based on Nvoc-protection and for comparison with previously known biological data of ³H-NPY. The in this way produced ³H-NPY was compared in binding assays to the commercially available ³H-NPY after photo chemical splitting off of the Nvoc-group and purification by HPLC.

a) Synthesis of N^{α}-Nvoc Protected NPY (Nvoc-NPY)

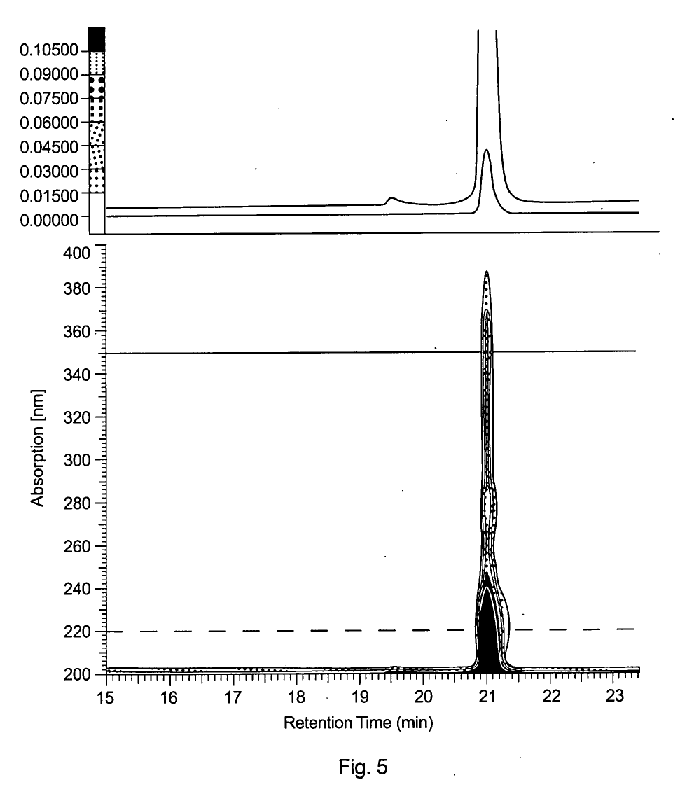

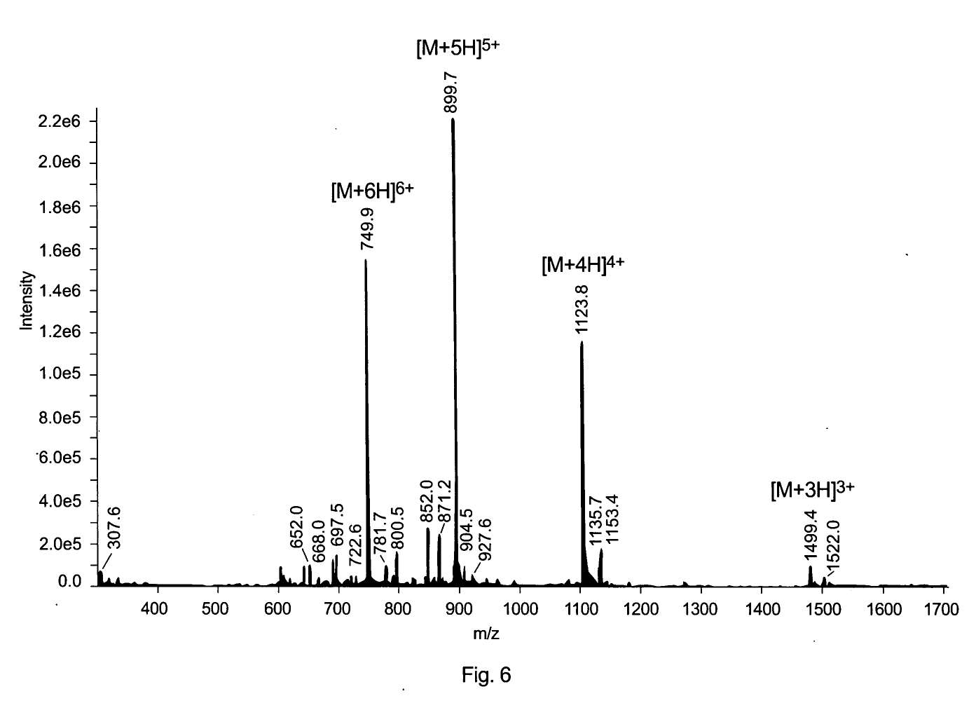

The NPY (molecule weight=4253.7) was produced by way of automatic solid phase peptide synthesis. The free N-terminal group of the peptide disposed on the polymer carrier was protected with the photo labile Nvoc-protective group following to the synthesis. In the following the peptide was split off from the resin. Here also the acid labile protective groups were removed. The raw peptide obtained in this manner was analyzed after preparative processing and was purified with preparative HPLC (FIG. 4). The presence of the Nvoc-protective group is recognizable in the DAD-spectrum by an absorption at 350 nm (FIG. 5). The calculated molecule mass of Nvoc-NPY (molecule weight=4492.9) could be confirmed by ESI-MS analysis illustrated in FIG. 6: m/z=749.9 [M+6H]⁶⁺ (calculated: 749.8), 899.7[M+5H]⁵⁺ (calculated: 899.6), 1123.8 [M+4H]⁴⁺ (calculated 1124.2) and 1499.4 [M+3H]³⁺ (calculated: 1498.6). Thus the desired product was present homogeneously and with a purity of better than 99% after the purification processing.

b) ³H-Marking of N^{α}-Nvoc NPY, Nvoc-Splitting Off and Purification

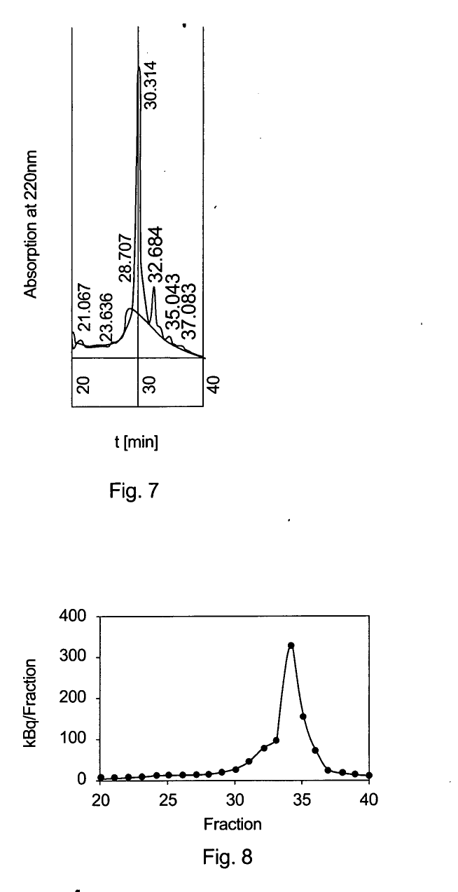

The free amino group of the Lys⁴-side chain of Nvoc-NPY (9 μg, 2 nmole) was derivatized by reaction with 100 μl N-succino-imidyl-[2,3-⁻³H] propionate (1 nmole, 3.7 MBq). A peak (Rₜ=32.7 minutes, fractions 31-36) could be isolated, wherein the retention time of the peak coincided with the retention time of “cold”-proprionylated NPY (FIG. 7) after irradiation with UV-light and following chromatographic separation of non-marked excess starting compound (wherein the retention time of the starting compound again corresponded to the retention time of the non-modified NPY after Nvoc-splitting off). The at Lys⁴-“cold”-proprionylated NPY was previously obtained at the resin after synthesis of Lys⁴(Dde)-NPY, splitting off of the Dde-protective group and by a following reaction propionic acid anhydride.

The radioactivity profile was determined in the β-counter by ³H-measurement of aliquots of the collected HPLC-fractions. A peak is recognizable at the fractions 31-36 in the HPLC-chromatogram and in the radioactivity profile, wherein the peak contains (22%) 0.8 MBq of radioactivity and thus corresponds (FIG. 8) to the desired radioactively marked peptide. The assumption can be made that the peptide is ³H-propionyl-Lys⁴-NPY.

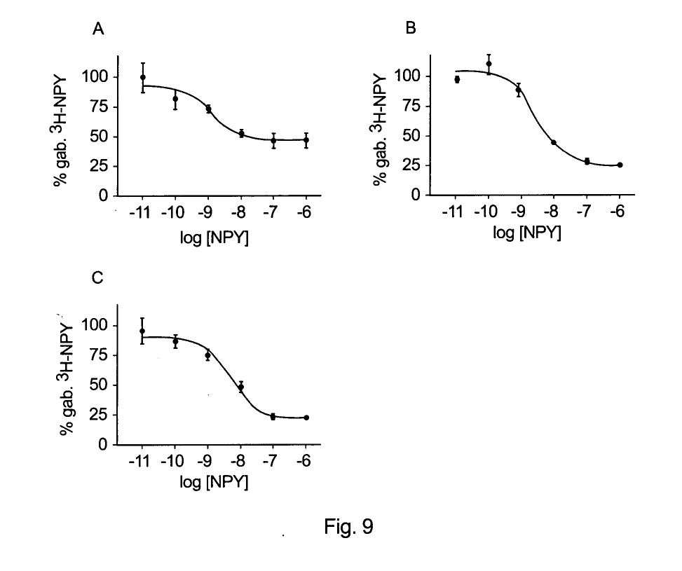

An identical behavior in comparison to commercially available and also at disposition marked ³H-NPY could be determined (FIG. 9) in competitive binding studies at the Y-receptors Y1, Y2, and Y5. The performance of the binding assays was performed as previously described.⁽⁵⁾

### EXAMPLE 2

### Preparation of ³H-proprionyl-Lys¹³-PTH(1-34)-amide (³H-PTH)

The para-thyroid-hormone (PTH) (1-34)-amide (molecule weight=4116.8) comprises in free-form three further amino groups in Lys-side chains (Lys¹³, Lys²⁶, and Lys²⁷) in addition to its N-terminal group. Lys¹³ was selected for marking, since importance in connection with the binding to the PTH-receptor is assigned to the N-terminal group and to the C-terminal region. (17)

a) Synthesis of N^{α}-and Lys^{26,27}-Nvoc-Protected PTH (1-34)-Amide (Nvoc-PTH)

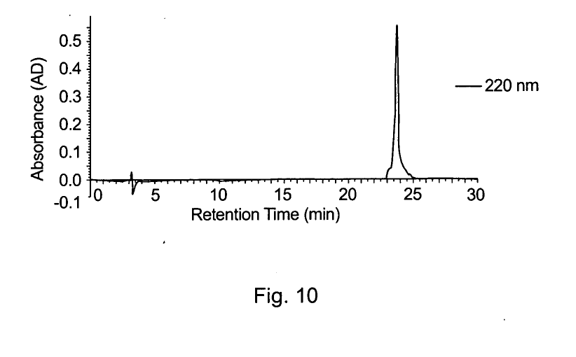

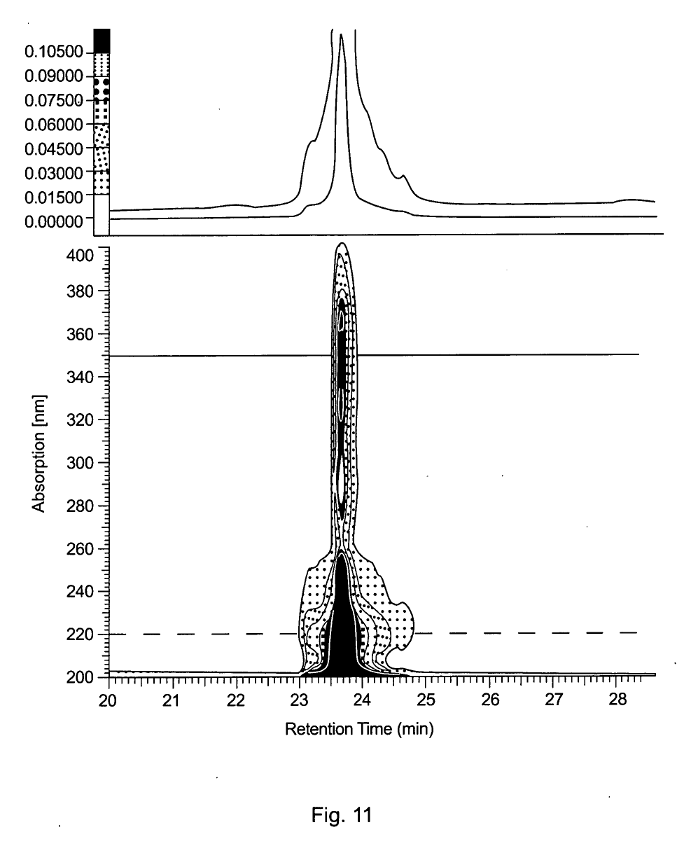

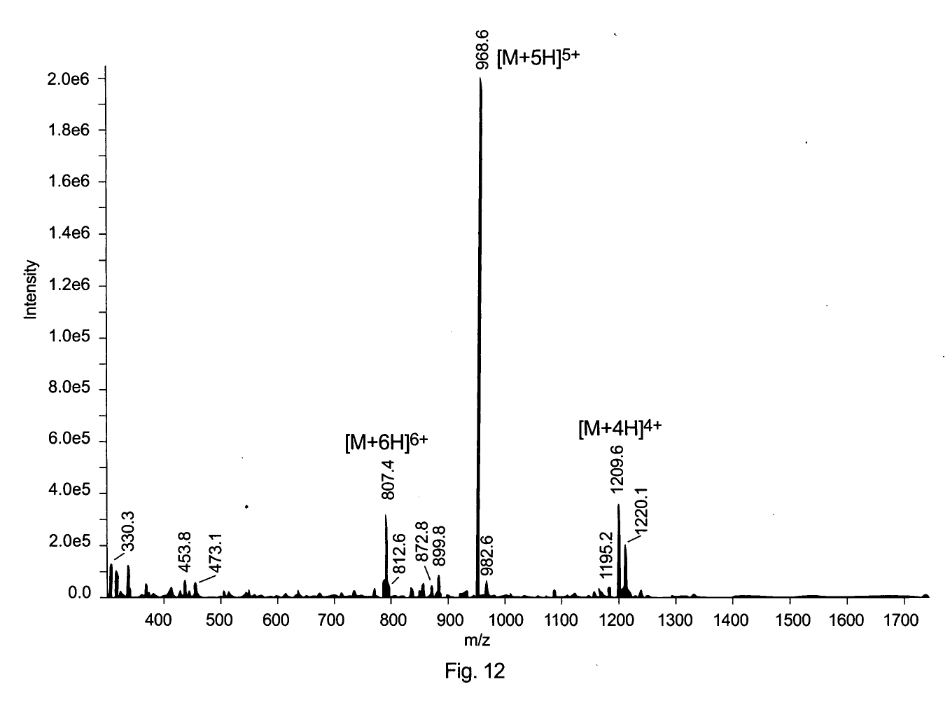

The synthesis of this peptide was performed with automatic solid phase peptide synthesis. Here Lys-derivatives protected by Dde protective groups were employed for the positions 26 and 27. The Lys^{26,27}-amino groups and the N-terminal group were simultaneously newly protected with the Nvoc-group after splitting off of the Dde-protective groups. A homogeneous product with a purity of more than 95% was present after splitting off and purification processing (FIG. 10). The presence of three Nvoc-groups of in the peptide (molecule weight=4836.4) could be confirmed by the DAD-spectrum and ESI-MS: m/z=807.4 [M+6H]⁶⁺ (calculated: 807.1), 968.6 [M+5H]⁵⁺ (calculated: 968.3) and 1209.6 [M+4H]⁴⁺ (calculated: 1210.1) (FIGS. 11 and 12).

b) ³H-Marking of N^{α} and Lys^{26,27}-Nvoc-Protected PTH (1-34)-amide, Nvoc-Splitting Off and Purification

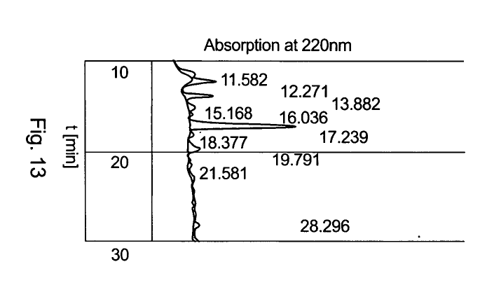

The free amino group of the Lys¹³-side chain of Nvoc-PTH (1-34)-amide (9.7 micrograms, 2 nmole) was reacted with N-succino-imidyl-[2,3-³H]-propionate in a borate buffer (pH=8.0). After irradiation with UV-light and following chromatographic separation from non-marked excess starting compound (wherein the retention time of the starting compound of 17.3 minutes after the Nvoc-splitting off corresponded again to the retention time of the unmodified PTH (1-34)-amide), a peak (Rₜ=19.8 minutes, fraction 19-23) could be isolated, wherein the retention time of the peak corresponded (FIG. 13) to the retention time of “cold”-proprionylated Lys¹³-PTH (1-34)-amide. The “cold” proprionylated PTH (1-34)-amide at Lys¹³ was obtained at the resin previously after synthesis of Lys¹³ (Dde)-PTH (1-34)-amide, splitting off of the Dde-protective group and by following reaction propionic acid anhydride.

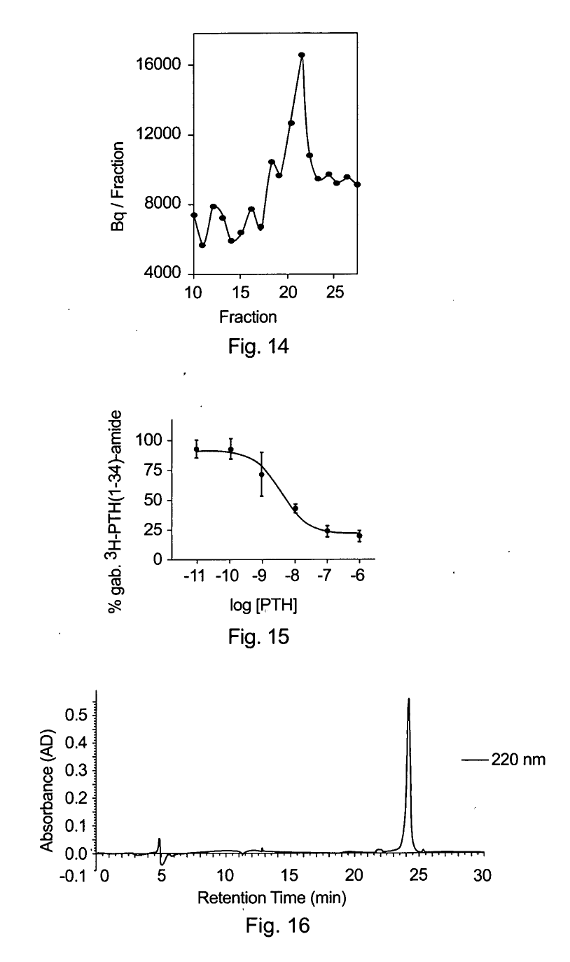

The radioactivity profile of the collected HPLC-fractions was determined by ³H-measurement of aliquots in a β-counter. A peak is recognizable at the fractions 19-23 in the HPLC-chromatogram and in the radioactivity profile, wherein the peak contains 58.9 kBq of radioactivity and corresponds to the desired radioactively marked peptide (FIG. 14). The assumption can be made that here ³H-propionyl-Lys¹³-PTH (1-34)-amide is present.

A nearly identical behavior relative to non-marked PTH (1-34)-amide could be shown (FIG. 15) in a competitive binding study at an PTH1-receptor based on an IC₅₀-value of 3.3 nmole at the radio ligand concentration of 1 nM. The performance of the binding assays was done as previously described.⁽⁵⁾ Therewith could be shown that the PTH (1-34)-amide marked at position 13 was marked selectively, is bio active and can be employed as a radio ligand.

### EXAMPLE 3

### Preparation of N-Terminal Nvoc-Protected Lys⁴-hPP

The human sequence of the pancreatic poly peptide (hPP) exhibits an amino group only through the N-terminal group. Since PP as NPY belongs to the same peptide family, a particular importance is also assigned to the N-terminal group of the PP in connection with the binding at the Y4-receptor. The N-terminal group of the PP should therefore be present in non-modified form in the design of the radio ligand. A Lys residue was introduced into the non-conserved position 4 during the synthesis of the peptide for this reason. A later radioactive marking is to be performed at position 4.

a) Synthesis of N^{α}-Nvoc-Protected Lys⁴-hPP (Nvoc-hPP)

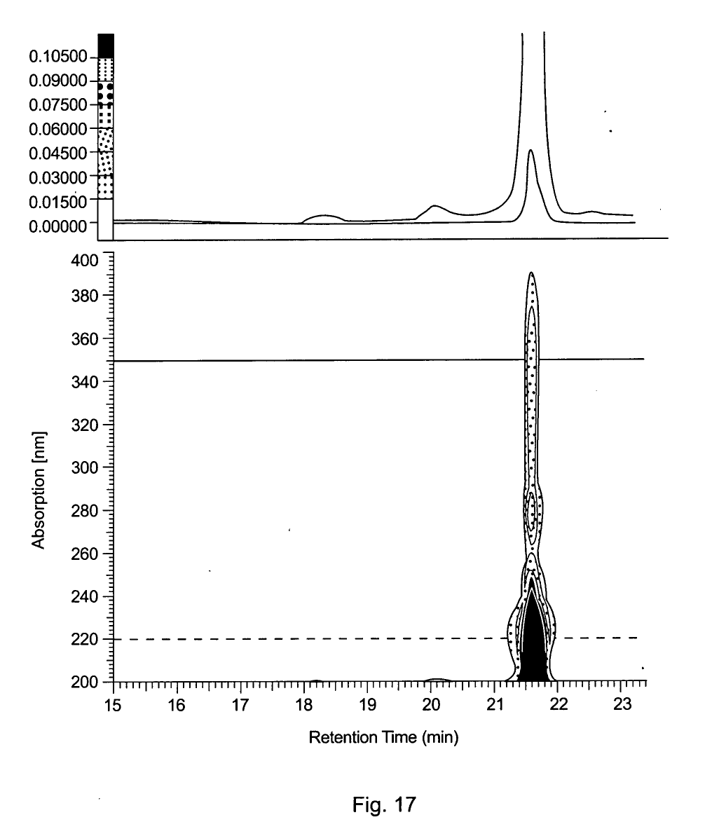

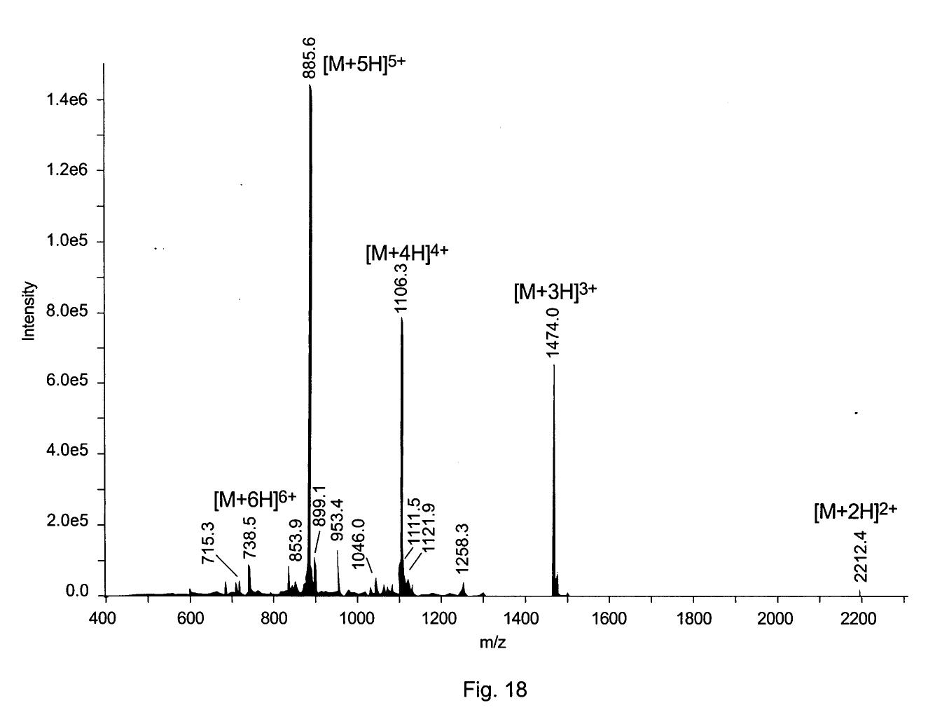

The N-terminal amino group of the peptide still remaining at the resident was again protected by the Nvoc-group after the synthesis of Lys⁴-hPP (molecule weight=4180.9). A homogeneous product with a purity of more than 95% was present after splitting off, preparative processing and purification with a preparative HPLC (FIG. 16). The presence of the Nvoc-group in the peptide (molecule weight=4421.1) could be confirmed by the absorption at 350 nm in the DAD-spectrum and ESI-MS: m/z=738.5 [M+6H]⁶⁺ (calculated: 737.9), 885.6 [M+% H]⁵⁺ (calculated: 885.2), 1106.3 [M+4H]⁴⁺ (calculated: 1106.3), 1474.0 [M+3H]³⁺ (calculated: 1474.7) and to 212.4 [M+2H]²⁺ (calculated: 2211.6). (FIGS. 17, 18).

### Definitions and Suggestions

Peptides are organic compounds comprising from 2 to about 100 amino acids. Peptides can be produced up to a length of 60 amino acids. α-, β-, γ-amino acids and α-, β-, γ-amino carbonic acids are employed for the synthesis. These acid can be polymerized in arbitrary combination. The thereby generated CO—NH connection is an amide bond and known as peptide bond. The peptides can have a C-terminal OH, NH₂, NHR (R=alkyl-), can contain alcohols or can be present acylated at the N-terminal group. Variations in the length and in the amino acid sequence of poly-peptides effect an unusual multitude of forms and of biological functioning of the peptides. Peptides are present in the complete cell region, wherein the spectrum of the physiological functions is very broad. Many peptides perform important functions in the regulation of the metabolism as hormones and are therefore of particular medical and pharmaceutical interest.

Table of abbreviations:

| | |
|---|---|
| 2-Cl-Z- | 2-Chlorobenzyloxycarbonyl |
| ACN | Acetonitrile |
| Bcc- | tert.Butoxycarbonyl- |
| DAD | Diode-array-detector |
| Dde- | 1-(4,4-dimethyl-2,6-dioxocyclohex-1-ylidene)ethyl- |
| DIC | N,N-Di-isopropyl-carbo-di-imide |
| DIEA | N,N-Di-isopropyl-ethyl-amine |
| DMF | N,N-Dimethylformamid |
| ESI-MS | Elektro-spray-mass-spectroscopy |
| Fmec- | N-(9-fluoro-enyl)methoxycarbonyl |
| ³H | Tritium |
| HOBt | 1-Hydroxy-benzotriazol |
| HPLC | High Performance Liquid Chromatography |
| ivDde- | 1-(4,4-dimethyl-2,6-dioxocyclohex-1-ylidene)-3-methylbutyl- |
| Mtt | 4-Methyltrityl- |
| NHS | N-Hydroxysuccino-imide |
| NPY | Neuropeptide Y |
| Nvec | Nitroveratryloxycarbonyl |
| PP | Pancreatic polypeptide |
| PTH | Para-thyroidal-hormone |
| tBu | tert.-Butyl |
| TFA | Tri-fluoro-acetic acid |
| Tfa- | Trifluoro-acetyl |
| UV | Ultra violet |
| Xas | individual amino acid |
| Z- | Benzyloxycarbonyl- |

Peptide Construction

In general all synthetically accessible peptides up to the recited upper maximum number of 60 amino acids can be constructed. Peptides with several amino groups are exclusively of interest for the marking strategy of the present application. Important as amino groups are here N-terminal (not acylated) and amino acids of the shape NH₂—(CH₂)ₙ—CH(NH₂)COOH (n=1-6). Here peptides are of interest have at least two free amino groups. These can be marked not only as was the case up to the present non-selectively, but according to the present Invention also selectively at selected amino groups. Peptides are here of concern wherein the peptide has more than one amino group.

Boc-Protective Group:

The amino group to be marked is Boc-protected during synthesis. An N^{ε}-Boc-protected Lys-derivative was employed for a marking of a Lys side chain during synthesis or, respectively, and an N^{α}-Boc-protected amino acid was employed for a marking of the N-terminal group. The Boc-protected derivatives are commercially available, such that the peptides do not have to be especially processed, in order to obtain the Boc-protective group.

The amino groups released free at a later point in time are Dde-protected.

This means that the amino groups which are present free after the marking reaction and the preparative processing are (initially) during the peptide synthesis protected at the polymeric carrier with the Dde group. The use of the Dde-protective group is described in the following literature:

Bettio, A.; Dinger, M. C.; Beck-Sickinger, A. G.: The neuropeptide Y monomer in solution is not folded in the pancreatic poly peptide fold, Protein Sci 2002, 11 (7), 1834-44. General descriptions relating to solid phase peptide synthesis can be found in: Jakubka, H.-D.: peptides, first edition, Spektrum Akademischer Verlag; Berlin, 1996

Chan W. C.; White, P. D.: Fmoc solid phase peptide synthesis, a practical approach, Oxford University press, 2000.

Protective Groups for Amino:

The following protective groups are possible for protecting the amino group: Boc, Dde, ivDde, Nvoc, Fmoc, Mtt, Z, Tfa, 2-Cl-Z.

Hosts:

A polymer carrier is employed for the builtup of the peptides for the solid phase peptide synthesis. This polymer carrier is designated as resin. The selection of commercially available polymer carriers and derivatives for the synthesis is large. Wang-resins can be employed for the synthesis of peptides with a free C-terminal acid. These resins comprise p-benzyl-oxy-benzyl-alcohol-functionalized polystyrene which is substituted with one percent di-vinyl benzene.

In most cases Rink-amide resin is employed for the synthesis of peptide amides. This is a 4-(2′,4′-dimethoxyphenyl-Fmoc-aminomethyl)-phenoxy-substituted polymer out of polystyrene with 1% divinylbenzene. Rinkamide resins were employed for the examples presented. These resins are available from Novabiochem und have a degree of substitution of 0.45 mmole/g.

Dde-Protective Group

The Dde-protective group was brought to the resin as a side chain protection of an Fmoc-protected Lys-residue (Fmoc-Lys(Dde)-OH) through a free carboxylic group. Such protected amino-acids are commercially available (for example from Novabiochem). Reference 8 describes the splitting off of this protective group.

Photo Labile

Photo labile means in connection with the present application that a reaction occurs under the influence of light (hv). Here light of a certain wavelength and strength is required.

The Nvoc-group is photo labile, since it is of the ortho-nitro-benzyl-type from a chemical point of view. These compounds are subject to a photo-enolization under the effect of light and a fragmentation occurs to ortho-nitro-benz-aldehydes.

All compounds of the ortho-nitro-benzyl-type can be employed as photo labile groups, which are split off with light hv at wavelengths from 200 to 500 nm during a time period of from 1 minute to 5 hours.

Simultaneousness

The splitting off of the Dde-group and the introduction of the Nvoc-group take place in the same reaction vessel, however, occur successively. Washing steps of the peptide disposed at the polymeric carrier and otherwise furnished with acid labile protective groups take place in between. In a first step the Dde-splitting off occurs at all respective amino groups in fact and in a following step also the Nvoc incorporation. The term simultaneous refers here to the amino acids.

Starting Materials

The Nvoc-protected peptides can also be obtained by employing of N^{α}-Fmoc and N^{α}-Nvoc-protected Lys-derivatives. Such derivative is presently not yet commercially available and has to be synthesized. The use of photo labile chemicals remains restricted to commercial chemicals and is performed only after completed peptide synthesis and in a simple way. The N-terminal group is also protected in the presented way during the same step.

No particular steps have to be taken during peptide synthesis, which in most cases occurs in the course of several days, such as a darkening of rooms and the like, such that the peptide synthesis can be performed under standard conditions.

Splitting Off from the Resin

The synthesis of the peptide is performed at the resin, individual amino acids are joined successively over several cycles to a chain. Therefore, the in this way synthesized peptide can be split off from the resin after performing the synthesis cycles.

Radio Active Marking

Also other NHS-esters with other isotopes or other activated radio active acyl residues can be employed, this results in a certain flexibility, which certainly is not undesired. The extension to other groups such as fluorescent dyes should be limited, since under certain circumstances these groups can bleach out under UV light.

The isotopes are for example ¹²⁵I; introduction is performed according to the Bolton-Hunter-reagent (2-(4-hydroxy-5-[¹²⁵I]-iodo-phenyl-)-propionyl-NHS-ester.

UV Light

UV lamps with a wavelength of from 200 to 500 nm can be employed as split-off conditions for the photo labile protective group. The strength of the power of the lamp can vary from about 10 to 1000 W. The splitting off can occur at a temperature between 0° C. and 30° C. within a time period of from 1 minute to 5 hours.

Purification

The purification is preferably performed with HPLC (high performance liquid chromatography). Since the marking never runs 100% and since in addition the Nvoc-peptide is employed in an excess relative to the radio active ester, there is required a separation of the peptide with derivative from the peptide without derivative. Since the two peptides differ in most cases only slightly with respect to their elution behavior depending on the transferred group, advantageously a gradient elution with two elution gradients was employed for separation. The gradient is depending under certain circumstances on the peptide sequence and on the quality of the column employed and therefore cannot be generalized for all sequences. The employed elution agents are: 0.08% t TFA in aceto-nitrile and 0.1% TFA in water.

An in detail description of HPLC of peptides can be found in the following literature:

Hencock, W. S.; Sparrow, J. T.: HPLC Analysis of biological compound, Vol 26, first edition, Marcel Dekker Inc. NEW York 1984.

Scintillation coctail

Solvent (toluene, xylene, Alkyl-naphthalene)

Primary scintillator (for example-phenyl);

Secondary scintillator (for example: 1,4-Bis(5-phenyl-oxazole-2-yl)-benzene or POPOP (p-bis(2-(5-phenyl-oxazolyl)benzene)).

Gels and/or emulsifying agents.

### LITERATURE

[1] Hulme, E. C., Receptor-Ligand Interactions: A Practical Approach. 1992, Oxford: IRL Oxford University Press.

[2] Rehm, H., Der Experimentator Proteinbiochemie/Proteomics. Vol. 3. 2000, Heidelberg: Spectrum-Verlag.

[3] Bolton, A. E. and Hunter, W. M., Labeling of proteins to high specific radioactivities by conjugation to an iodine-125-containing acylating agent. Application to the radiommunoassay. Biochem. J., (1973) 133, 529-538

[4] Vincent, J. P. and Gaudriault, G., The importance of pH in labeling proteins and peptides by acylation of their α-amino groups with a reactant carrying an activated carboxylic group. (1993) in FR 2687680: Frankreich.

[5] Cabrele, C. Wieland. H. A., Koglin. N., Stidsen, C. and Beck-Sickinger, A. G., Ala(31)-Aib(32): identification of the key motif for high affinity and selectivity of neuropeptide Y at the Y(5)-receptor. Biochemistry, (2002) 41, 8043-9.

[6] Pillai, V. N. R., Photoremovable protecting groups in organic synthesis. Synthesis, (1980) 1, 1-25

[7] Dorman. G. and Prestwich, G. D., Using photolabile ligands in drug discovery and development. Trends Biotechnol, (2000) 18, 64-77.

[8] Fodor. S-P., Read, J. L., Pirrung, M. C., Stryer, L., Lu, A. T, and Solas, D., Light-directed, spatially addressable parallel chemical synthesis. Science, (1991) 251, 767-73.

]9] Bycroft, B. W., Chan, W, C., Chhabra, S. R., Teesdale-Spittle, P. H. and Hardy, P. W. A novel amino protection-deprotection procedure and its application in solid phase peptide synthesis. J. Chem. Soc., Chem. Commun., (1993) 9, 776-777

[10] Rusiecki, V. K. and Warne, S. A., Synthesis of Nα-Fmoc-Nε-Nvoc-lysine and use in the preparation of selectively functionalized peptides. Bioorg. Med. Chem. Lett., (1993) 3, 707-710

[11] Kaiser, E, Colescott, R. L., Bossinger, C, D: and Cook, P. I., Color test for detection of free terminal amino groups in the solid phase synthesis of peptides. Anal. Biochem., (1970) 34, 595-598

[12] Beck, W. and Jung, G., Convenient reduction of S-oxides in synthetic peptides, lipopeptides and peptide libraries. Lett. Pept. Sci., (1994) 1, 31-37

[13] Stefanowicz, P. and Siemion, I. Z., Reactivity of N-hydroxysuccinimide esters. Pol, J. Chem., (1992) 66, 111-118

[14] Tang, Y. S. Davis, A. M. and Kitcher, J. P., N-Succinimidyl propionate: characterization and optimum conditions for use as a tritium labeling reagent for proteins. J. Labelled Compd. Radiopharm., (1983) 20, 277-284

[15] Cabrele, C. and Beck-Sickinger, A. G., Molecular Characterization of the Ligand-Receptor Interaction of the Neumpeptide Y Family. J. Peptide Sci, (2000) 6, 97-122

[16] Chang, R. S., Lotti, V. J., Chen, T. B. Cerino, D. J. and Kling, P. J., Neuropeptide Y (NPY) binding sites in rat brain labeled with ¹²⁵I-Bolton-Hunter NPY: comparative potencies of various polypeptides on brain NPY binding and biological responses in the rat vas deferens. Life Sciences, (1985) 37, 2111-2122

[17] Juppner, H., Schipani, E., Bringhurst, F. R., McClure, I., Keutmann. H_T., Potts, J. T., Kronenberg, H. M, Abou-Samra, A. B., Segre, G V. and Gardella, T. J., The extracellular amino-terminal region of the parathyroid hormone (PTH)/PTH-related peptide receptor determines the binding affinity for carboxyl-terminal fragments of PTH-(1-34). Endocrinology, (1994) 134, 879-884

1. Method for selective radioactive marking of amino groups in peptides, wherein a peptide is built up at a carrier material by chemical solid phase peptide synthesis from amino acid components characterized in that an amino acid component is incorporated at at least one position to be marked, wherein the amino acid component carries an amino group, wherein the amino group is protected by a group which distinguishes functionally from protective groups at amino groups not to be marked, wherein the protective group is selectively removed from the position to be marked and wherein the peptide is dissolved from the carrier material and the thereby obtained selectively de-protected peptide is reacted in solution with radioactively marked amino reactive substances, and wherein in the following protective groups are split off at the amino acid components not to be marked and the thereby obtained selectively radioactive marked peptide is purified in a conventional manner.

2. Method according to claim 1 characterized in that the not to be marked amino groups carry hydrazine labile protective groups during synthesis.

3. Method according to claim 1 characterized in that the amino groups not to be marked carry photo labile protective groups during synthesis.

4. Method according to claim 1 characterized in that the protective groups of all amino groups not to be marked are initially selectively removed and the free amino groups are new protected with photo labile protective groups.

5. Method according claim 1 characterized in that the amino groups to be marked carry acid labile protective groups during synthesis.

6. Method according to claim 2 characterized in that the hydrazine labile protective groups are Dde-groups.

7. Method according to claim 3 characterized in that the photo labile protective groups are Nvoc-groups.

8. Method according to claim 5 characterized in that the acid labile protective groups are Boc-groups.

9. Method according to claim one wherein NHS-esters are employed as amino reactive substances for radioactive marking.

10. Method according to claim one characterized in that N-succino-imidyl-[2,3-³H]-propionate is employed as NHS-ester.

11. Method according to claim 1 characterized in that the radioactive marking is performed with an excess of peptide and at room temperature.

12. Method according to claim 1 characterized in that cooling is performed after the marking, acidification is performed with TFA and irradiation with UV light at 366 nm occurs for 30 to 60 minutes for splitting off of the photo labile protective groups.

13. Method according to claim 1 further comprising
protecting all amino groups not to be marked with protective groups;
leaving unprotected at least one amino group to be marked for production of a selectively radioactively marked radio peptide.

14. Method according to claim 1 further comprising
protecting all amino groups not to be marked with photo labile protective groups;
leaving unprotected at least one amino group to be marked for production of a selectively radioactive marked peptide.

15. Method for selective radioactive marking of peptides characterized in that initially a peptide is constructed out of commercially available Fmoc/tBu-protected amino acids,
wherein the later to be marked amino group is Boc-protected,
whereas the later free amino groups of the side chains are Dde-protected during the synthesis,
wherein the Dde-protective groups at the resin are split off with hydrazine, simultaneously the now free amino groups are furnished with the photo labile protective group nitro-veratryl-oxy-carbonyl—Nvoc—,
the peptide is split off from the resin and is purified,
the radioactive marking is performed with NHS-esters,
the photo labile protective groups are split off by interaction with UV light, and
the obtained selectively marked peptide is purified by HPLC in a way known in principle.

16. Method for selective radio marking of peptides according to claim 15 characterized in that the peptide is disposed in solution during the marking phase.

17. Method for selective radio marking of peptides according to claim 15 characterized in that lysine with Dde-protective groups is employed for peptide buildup.

18. Method for selective radio marking of peptides according to claim 15 characterized in that the radioactive marking is performed with NHS-esters such that peptide dissolved in a buffer is added in excess of the ester and is maintained for time period of from 1 to 2 hours at room temperature.

19. Method for selective radio marking of peptides according to claim 15 characterized in that the cold charge is radiated for a time period of 30 to 60 minutes at 366 nm with UV light after acidification with TFA.

20. Method for selective radio marking of peptides according to claim 15 characterized in that the esterification is performed with N-succino-imidyl-[2,3-³H]-propionate.
